(19) United States
(12) Patent Application Publication
(10) Pub. No.: US 2020/0289134 A1
(43) Pub. Date: Sep. 17, 2020

(54) TISSUE CORING DEVICE

(71) Applicant: MFr Technologies, Inc., Maple Grove, MN (US)

(72) Inventors: Trevor Jacob Laughlin, Minnetonka, MN (US); Joseph Jude Saladino, Eden Prairie, MN (US); Daniel M. Fisher, Maple Grove, MN (US); Jason L. Koh, Winnetka, IL (US)

(21) Appl. No.: 16/815,908

(22) Filed: Mar. 11, 2020

Related U.S. Application Data
(60) Provisional application No. 62/816,699, filed on Mar. 11, 2019.

(51) Int. Cl. A61B 17/16; A61B 10/02

(52) U.S. Cl. CPC A61B 17/1637; A61B 2010/0258; A61B 2010/0208; A61B 10/025

(57) ABSTRACT

An automated tissue coring device that enables controlled and repeatable removal of bone and bone marrow. The device includes a handle, a biasing element coupled to an advancement mechanism, an actuator, and a hollow penetrating needle. A method for bone and bone marrow biopsy, bone marrow aspiration, and bone marrow enhanced tissue repair are introduced using the device as described herein.

### CROSS-REFERENCE TO RELATED APPLICATION

The present application claims the benefit of U.S. Provisional Application No. 62/816,699 entitled “SURGICAL INSTRUMENTS AND METHODS”, filed Mar. 11, 2019, which is incorporated herein by reference in its entirety.

### TECHNICAL FIELD

The present invention is generally directed to devices for medical procedures, and more particularly, for bone marrow access.

### BACKGROUND OF THE INVENTION

Healthcare professionals occasionally access bone marrow for trephine biopsies, bone marrow aspirations or bone marrow enhanced tissue repair. Bone marrow aspirations and trephine biopsies are often performed or directed by interventional radiologists and hematopathologists, while bone marrow enhanced tissue repair procedures are often performed by orthopedic surgeons.

In a bone marrow aspiration, a hole is created at the biopsy site, a needle is inserted, and liquid blood and bone marrow are withdrawn. Blood and bone marrow cells are examined and checked for blood disorders, chromosome problems, and infection.

In a bone marrow trephine procedure, a core of bony anatomy is removed from the patient so that the bone marrow structure can be observed in order to evaluate the health of the patient and diagnose cancers such as lymphoma, leukemia, and myeloma. Bone marrow aspirations and bone marrow trephines are most often performed together, with the aspiration first, followed by the trephine extraction.

The second popular example use of the present invention is stimulating bone marrow in subchondral bone to repair cartilage. Bone marrow is rich in stem cells, which have great healing and regenerative properties. Other examples include the stimulation of healing in areas of tendinosis such as elbow lateral epicondylitis, patella tendinopathy, hip gluteus medius tendinopathy, and ankle Achilles tendinitis; to stimulate ligament healing such as in knee medial collateral ligament sprains; to enhance soft tissue to bone healing such as in the repair of the shoulder rotator cuff tendon to bone; for the enhancement of bony healing in fractures; and in the preparation of bone for improved healing to prosthetic implants

Bone marrow and its associated cells are known to have regenerative properties which makes it valuable medicinally in areas of wear, damage, or impairment. In many cases, soft tissue and bone healing can benefit from improved access to bone marrow, typically through small holes in bone. One area of benefit is in articular cartilage repair. Articular cartilage is a smooth, low-friction tissue which covers the ends of bones and enables healthy joint function. Articular cartilage is prone to damage from excessive wear or traumatic injuries, as are common in sports. When articular cartilage is damaged, it can result in pain and reduced mobility for the patient, and in some cases subsequent arthritis. Articular cartilage has extremely limited ability to repair itself spontaneously due to absent blood flow.

Microfracture surgery exists as a method to assist in the repair of articular cartilage in order to improve joint function. Microfracture creates a pathway for cartilage-building cells in blood and bone marrow to travel from the underlying cancellous bone to the articular surface by producing small holes in the cortical bone. Microfracture procedures are typically performed using an awl or a pick that is hit with a hammer.

Other conditions where healing is often limited or impaired occurs in degenerative conditions where soft tissue attaches to bone, such as in rotator cuff tears and various insertional tendinopathies such as elbow lateral epicondylitis, patella tendinopathy, and Achilles tendinitis. In these situations, there is again limited or absent blood flow, and therefore healing is impaired without access to the necessary cells and growth factors.

Drilling or perforation of the bone is performed to allow bone marrow and blood to access the area of damage. Similarly, in cases of delayed or absent fracture healing, or in the preparation of surfaces for bone to implant healing, drill holes are often made to allow bone marrow and blood to reach the area of relatively poor circulation. Some marrow access devices, for example U.S. Pat. No. 9,510,840 (“the '840 patent), are utilized via driving a wire with a hammer through an angled cannula. Like the hammer and awl method, this method requires a minimum of three hands to operate and delivers inconsistent results due to its subjective and uncontrolled external force delivery, which is a problem in microfracture procedures. Reported clinical results of microfracture are very good in some cases, but other researchers have reported relatively poorer results. Part of this may be related to the variability of the manually performed technique. To improve effectiveness during procedures, active surgeon feedback such as good visibility is of prime importance. Many marrow access procedures are performed arthroscopically. Operating a surgical scope (arthroscope) requires focus, precision, and a steady hand, and the coordination of meticulous hole creation relies upon such control. Therefore the primary operator is often inclined to maintain control of the scope. This leaves the primary operator's other hand available for one of two tasks: Hold the awl, or swing the hammer. Both of these require equal or higher levels of finesse to operate effectively, and are interdependent from one another and from the scope.

To date, the typical method of performing microfracture involves holding a longitudinal awl with an angled tip, and a hammer for impacting the proximal end of the handle of said awl. At the same time, a surgical scope must be held and positioned in a manner which allows the surgeon to see the tip alignment, the depth of penetration, and the subsequent blood flow from each hole produced. As such, a problem exists in that at least three hands are required to perform such a procedure using the historically accepted method. While each tool must be operated with careful precision, and the feedback from each tool is interdependent, coordinating a microfracture procedure with a minimum of two operators presents a challenge.

There are several technical challenges associated with the creation of microfracture holes in the bone. The depth of penetration must be sufficient to adequately access the bone marrow elements underneath the relatively avascular subchondral bone. The holes must be of sufficient width to allow bone marrow and blood to reach the surface of the bone, while not being so large as to significantly affect the load-bearing characteristics of the bone. Holes must be adequately spaced apart to allow for adequate flow to cover the surface, but not collapse into each other. Ideally, the holes should be perpendicular to the surface so that minimal tissue is perforated to allow access to the bone surface.

The standard technique uses a hammer manually impacting the back end of the awl. This can result in a highly variable amount of force being applied, resulting in unpredictable hole size and depth. In addition, excessive load can cause significant bone edema, pain and loss of function in patients. Furthermore, the direction of force applied by the hammer is not substantially aligned with the orientation of the tip, and the tip may not be perpendicular to the bone surface. This often results in substantial undesired damage to the subchondral bone, since an oblique hole or trough may be created.

In many cases, the lateral force transmitted to the awl tip causes the tip to break into an adjacent hole, significantly disrupting the subchondral bone. In other cases, the individual holes created may be much wider than what is necessary, leading to complications and prolonged recovery time. There are also multiple awl types, sizes, and tip designs. Many of these designs have very thick and robust tips to withstand the obliquely applied hammering force, but this can create issues with size of hole creation. In addition, the majority of these instruments are multiple-use, and tend to dull or blunt over time, resulting in a need for increased force application to create the holes.

Another example application of the present invention is to improve access to bone marrow and blood to enhance soft tissue or bony healing, including fracture union, fusion, or healing to prosthetic implants. Insufficient access to bone marrow in said procedures can result in reduced progenitor cells and growth factors, and ultimately substandard clinical outcomes. Currently, this access is achieved either with the use of an awl, with the previously described deficiencies; or by drilling into the bone.

Drilling of the bone has several limitations: typically, this is performed through an open and not minimally invasive surgical technique. The angle of drilling is usually limited by use of a straight drill bit. Larger holes can weaken the underlying bony tissue, while smaller drill bits are prone to breakage due to the often awkward positioning and unbalanced size of the power drill. Drilling has also been implicated in thermal necrosis (death) of the bone, which is counterproductive in the healing environment. This can be exacerbated by the typical reuse of many drill bits which become duller with continued use. Finally, drilling with the typical size drill and bit is usually a two-handed procedure requiring an assistant to retract adjacent tissue.

### SUMMARY OF THE INVENTION

The present invention introduces a novel instrument for use in microfracture procedures and other bone marrow access procedures which solves the multiple issues mentioned above.

The novel instrument can be operated using one hand, emulating both the hammer and the awl of the historically accepted microfracture procedure, or the stabilized drill and bit. In such form, one operator may coordinate each essential surgical element simultaneously with precision.

Additionally, the device can have variable angles to access the bone, unlike a straight awl or drill bit. The present invention demonstrates a means of transmitting power to a force in a direction better aligned with the orientation of the tip. This device can deliver a precise load and direction to the tip, resulting in much better controlled hole size, shape, and depth.

Another advantage is a disposable tip, which can also increase the average sharpness of the instrument when used.

The present invention comprises a one-handed solution for creating holes in tissue. In one embodiment of the present invention, the entire device is disposable, so as to ensure a safe and sterile procedure administered by the device. In another embodiment, the tip is removable, and can be cleaned by standard reprocessing methods.

In yet another embodiment, the present invention includes a handheld surgical instrument having an energy storage element, wherein the energy storage element is a spring coupled to the impacting mechanism, the impacting mechanism having a tip configured to impact a bone, wherein the tip includes a tapered point, a power transmission mechanism is configured to transmit energy from the energy storage element to the impacting mechanism, wherein the power transmission mechanism includes a semi flexible metal wire guided by a hollow shaft, wherein the hollow shaft includes a distal end, wherein the semi-flexible metal wire includes a bend toward the distal end. A trigger mechanism is configured to release energy from the energy storage element, wherein the bend includes an angle, wherein the trigger mechanism includes a manual lever which, when actuated, simultaneously retracts the tip and charges the energy storage element.

In an alternative embodiment, the invention includes a method of performing surgery that includes the use of a handheld surgical instrument comprising an energy storage element, wherein the energy storage element is a spring coupled to the impacting mechanism. An impacting mechanism has a tip configured to impact a bone, wherein the tip includes a tapered point. A power transmission mechanism is configured to transmit energy from the energy storage element to the impacting mechanism, wherein the power transmission mechanism includes a semi-flexible metal wire guided by a hollow shaft, wherein the hollow shaft includes a distal end. The semi-flexible metal wire includes a bend toward the distal end. A trigger mechanism is configured to release energy from the energy storage element, wherein the bend includes an angle, wherein the trigger mechanism includes a manual lever which, when actuated, simultaneously retracts the tip and charges the energy storage element.

The present invention is an automated tissue coring device which comprises a handle, a hollow shaft, a biasing element, a first actuator, and a hollow penetrating needle with an elongate portion and a sharp tip. The distal portion of the hollow shaft is optionally curved for increased access in minimally invasive procedures. The handle houses a biasing element, optionally configured to an impactor and an indexing mechanism, which enables a user to apply a known force and/or advance a known distance, the penetrating needle tip upon triggering a first actuator. Biasing element may be coupled with one or more linear advancement mechanisms, such as the following list, for example and not limitation: a linear indexing Geneva mechanism, a rack and pinion, a slider crank, a barrel cam and follower, a slotted bar quick return mechanism, a Whitworth mechanism, a vibrating penetrator, an oscillating swash plate mechanism.

The penetrating needle tip may be retracted through the use of an optional second actuator, an optional toggle, a secondary mode of the first actuator, or some combination thereof. By way of example and not limitation, a secondary mode of the first actuator may be enabled by pushing the first actuator forward, thereby mating an internal catch between the first actuator and the hollow needle as made by one with ordinary skill in the art, and then pulled back to retract the needle. Hollow shaft and its internal components are optionally removable from the handle and exchangeable with other attachments. In one example embodiment, system includes a first configuration for aspiration comprising a hollow tube with a port for suction and housing a solid removable elongate slider with cutting tip; and a second configuration for coring, comprising a hollow needle and a second elongated slider for column support. In this example embodiment, the hole in the cortical bone layer is created with first elongate slider, elongate slider is removed from hole and suction is applied to extract liquid blood and bone marrow, first elongate slider is removed from the handle out of the proximal side and replaced with a hollow needle and second elongate slider while keeping the hollow shaft in place, and then the needle tip is advanced beyond the bone surface to achieve sufficient depth for retrieving a core sample.

The present invention includes an automated tissue coring device which may be utilized for bone marrow biopsies. In one embodiment, a bone marrow biopsy is performed with the following steps: Place the distal tip at the site of interest; second charge the internal spring or biasing element by squeezing the lever or first actuator. Apply tip pressure to the bone surface and actuate the first impact or advancement by squeezing the trigger or second actuator. Trigger impact or advancement again if desired depth is not yet achieved. Depth may be indicated by markings on the needle or via a window in the handle which shows how far the needle has moved from its starting position. Repeat triggering impact or advancement until desired depth is achieved. With the hollow shaft tip in place, pull back on the handle toggle to retract the needle from the bone. Remove the penetrating needle with the core in it from the back of the device. Attach provided tight sealing fitting onto the adapter, sealing off the proximal-most section of the hollow shaft, and creating a continuous channel between the fitting and the end of the shaft. With the hollow shaft tip tightly in place at the entrance to the hole, apply suction to the fitting in order to withdraw blood and bone marrow. A second hollow elongated body may be inserted through the back of the device before the fitting is attached in order to seal off the proximal section and taper down the distal end for a tight seal on the hole in the bone. Finally, extract the bone tissue core from the needle by pushing it through a wire opening with an elongated slider. In another embodiment, the automated tissue coring device of the present invention may be utilized for bone marrow enhanced tissue repair. Bone marrow stimulation is achieved by performing the following detailed steps: Place the distal tip at the site of interest; second charge the internal spring (biasing element) by squeezing the lever (first actuator). Apply tip pressure to the bone surface and actuate first impact or advancement by squeezing the trigger (second actuator). Trigger impact or advancement again if desired depth is not yet achieved. Depth may be indicated by markings on the needle or via a window in the handle which shows how far the needle has moved from its starting position. Repeat triggering impact or advancement until desired depth is achieved. Once desired depth is achieved, engage the toggle on the handle to activate a retraction mode, and then squeeze the second actuator to retract the needle. When the actuator reaches the end of its stroke the toggle is pushed back into forward mode. Repeat from step until desired number of holes are created in bone. Each new hole will drive cores from previous holes further proximally into the hollow penetrating needle. In an alternative embodiment an elongated slider is used after each coring operation to press out and dispose of the core in order to provide an unobstructed needle opening for the next coring operation.

These and various other characteristics are pointed out with particularity in the claims annexed hereto and form a part hereof. Reference should also be made to the drawings which form a further part hereof, and to accompanying descriptive matter, in which there are illustrated and described representative examples of systems, apparatuses, and methods.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIG. 1 depicts a side view of the automated tissue coring device.

FIG. 2 depicts a cross-section of a hollow needle inside a hollow shaft positioned at a bone surface.

FIG. 3 depicts a hollow needle tip as advanced a known distance with respect to a hollow shaft.

FIG. 4 depicts a hollow needle tip as advanced a known distance twice with respect to a hollow shaft.

FIG. 5 depicts a method of automated tissue transfer whereby both solid bone and liquid bone marrow are obtained from a site for removal or transfer.

FIG. 6 depicts a hollow penetrating needle cross-section with a core retention element.

FIG. 7 depicts a blood or bone marrow aspiration device whereby a hole is created and negative pressure is utilized to draw out fluid.

FIG. 8 depicts a suction mechanism for drawing regenerative cells out of bone with a multi-lumen tube.

FIG. 9 depicts a block diagram illustrating a biopsy method using the present invention.

FIG. 10 depicts a block diagram illustrating a marrow stimulation method using the present invention.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION OF THE DRAWINGS

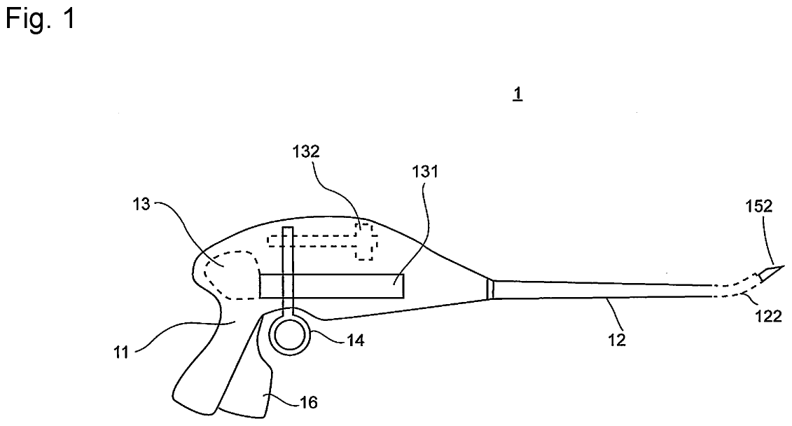

As presented in FIG. 1, an automated tissue coring device 1 comprises a handle 11, a hollow shaft 12, a biasing element 13, a first actuator 14, and a hollow penetrating needle with an elongate portion and a sharp tip 152. The distal portion 122 of the hollow shaft is optionally curved for increased access in minimally invasive procedures. The handle houses a biasing element 13, optionally configured to an impactor and an indexing mechanism 131, which enables a user to apply a known force and/or advance a known distance, the penetrating needle tip upon triggering a first actuator 14. Biasing element may be coupled with one or more linear advancement mechanisms, such as the following list, for example and not limitation: a linear indexing geneva mechanism, a rack and pinion, a slider crank, a barrel cam and follower, a slotted bar quick return mechanism, a whitworth mechanism, a vibrating penetrator, an oscillating swash plate mechanism. The penetrating needle tip may be retracted through the use of an optional second actuator 16, an optional toggle 132, a secondary mode of the first actuator, or some combination thereof.

By way of example and not limitation, a secondary mode of the first actuator may be enabled by pushing the first actuator forward, thereby mating an internal catch between the first actuator and the hollow needle as made by one with ordinary skill in the art, and then pulled back to retract the needle. Hollow shaft and its internal components are optionally removable from the handle and exchangeable with other attachments. In one example embodiment, system includes a first configuration for aspiration comprising a hollow tube with a port for suction and housing a solid removable elongate slider with cutting tip; and a second configuration for coring, comprising a hollow needle and a second elongated slider for column support. In this example embodiment, the hole in the cortical bone layer is created with first elongate slider, elongate slider is removed from hole and suction is applied to extract liquid blood and bone marrow, first elongate slider is removed from the handle out of the proximal side and replaced with a hollow needle and second elongate slider while keeping the hollow shaft in place, and then the needle tip is advanced beyond the bone surface to achieve sufficient depth for retrieving a core sample.

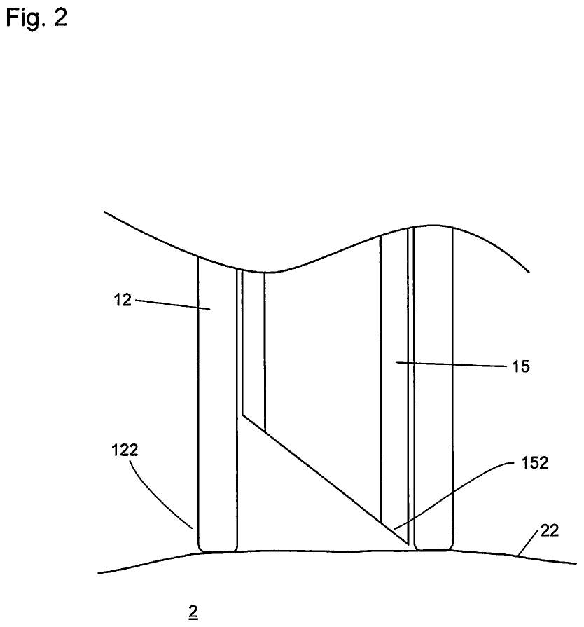

As presented in FIG. 2, the distal tip of the device, and more particularly the distal tip 122 of the hollow shaft 12, is positioned against the bone surface 2 at the site of interest. Before advancement of the hollow needle 15 has been actuated, the sharp tip 152 is located at or proximal to the bone surface 22 in the primary embodiment.

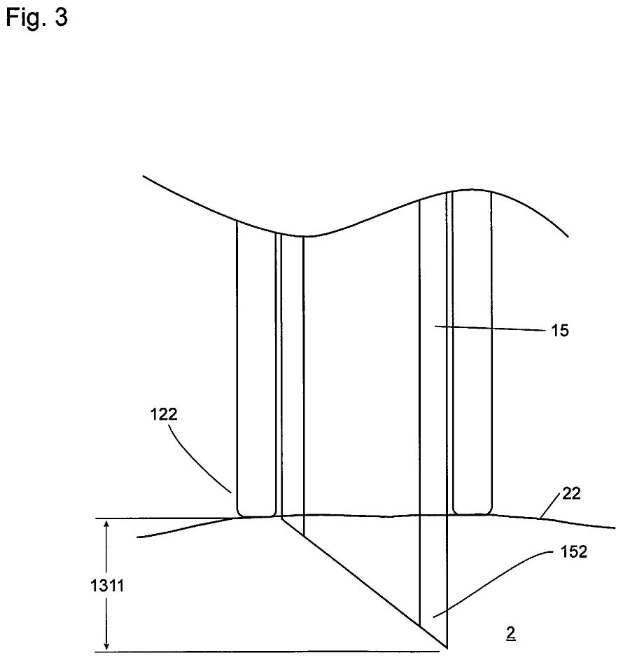

As presented in FIG. 3, the distal tip of the device, and more particularly the distal tip 122 of the hollow shaft 12, is positioned against the bone surface 22 at the site of interest. After one user actuation, and associated advancement of the hollow penetrating needle 15 via the biasing element, the sharp tip is advanced a known distance, 1311, past the surface of the bone 22, and into the underlying bone 2, which contains blood and bone marrow. Alternatively, the needle tip may be advanced with a known force, and at a visibly measurable distance. Furthermore, said known force could be adjusted by the user if needed. By way of example and not limitation, known force may start relatively small, and if denser than normal bone is encountered, a user may adjust a dial on the handle housing, resulting in greater spring deformation, and more force output.

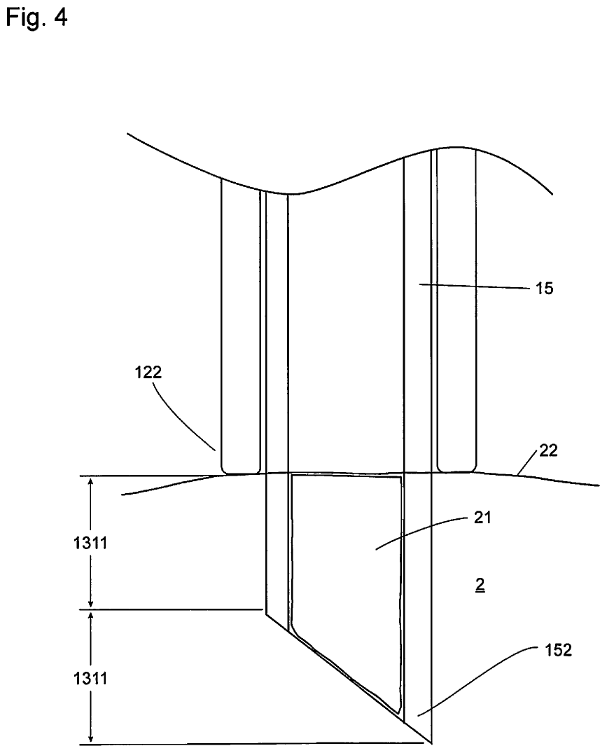

As presented in FIG. 4, the distal tip of the device, and more particularly the distal tip 122 of the hollow shaft 12, is positioned against the bone surface 22 at the site of interest. After two user actuations, and associated advancements of the hollow penetrating needle 15 via the biasing element, the sharp tip is advanced twice a known distance 1311, past the surface of the bone 22, and into the underlying bone 2, which contains blood and bone marrow. Actuation of needle tip advancement may be repeated until desired depth is reached for core 21 extraction. Advancing the needle tip in an incremental fashion allows the user to precisely control the outcome, gathering feedback after each actuation. Alternatively, the needle tip may be advanced to a predetermined final depth upon first actuation, leveraging momentum and force delivered from the biasing element. The velocity of such a mechanism has advantages in shearing spongy bone without crushing and deforming the core so as to negatively affect diagnosing.

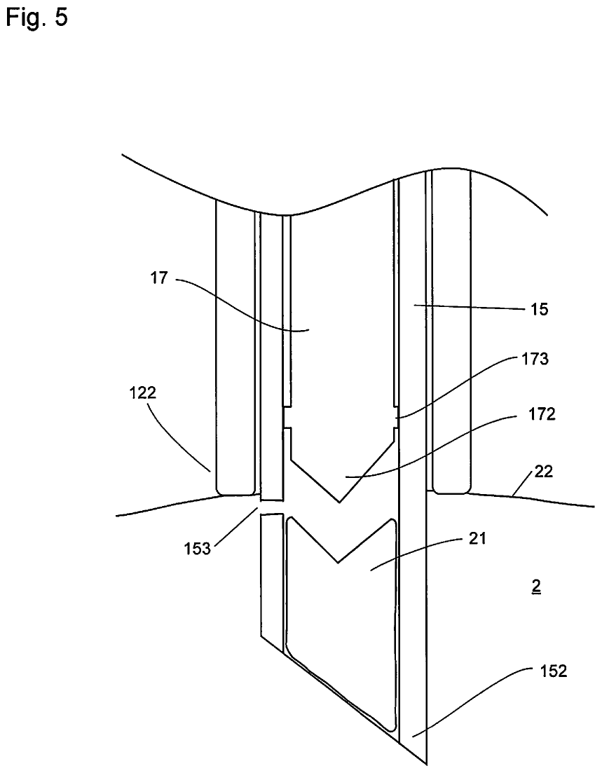

As presented in FIG. 5, a hollow needle 15 is positioned within a hollow shaft 12 and around an elongate slider 17. The elongate slider and the hollow shaft together support the hollow penetrating needle. The needle may comprise additional openings 153 for liquid blood and bone marrow flow. The hollow needle depicted is driven beneath the bone surface to desired depth for removing a core 21. Proximal end 172 of elongated slider may be configured with a cutting tip for penetrating cortical bone surface 22. In one embodiment, needle and elongated slider are driven into bone together until the cortical layer is penetrated, and then separated such that the needle may be advanced further into bone 2 until sufficient depth is achieved. Suction may be applied proximally within the hollow shaft 15 using an internal plunger 173 or an external negative pressure element. In one embodiment, the penetrating needle is configured as a multi-lumen tube such that one lumen captures a solid core, and another lumen captures liquid blood and bone marrow when negative pressure is applied. After the hollow needle is retracted from the site, taking with it the tissue of interest, the elongated slider may be used to eject the core into a second site or outside the body by being pushed distally with respect to the hollow shaft.

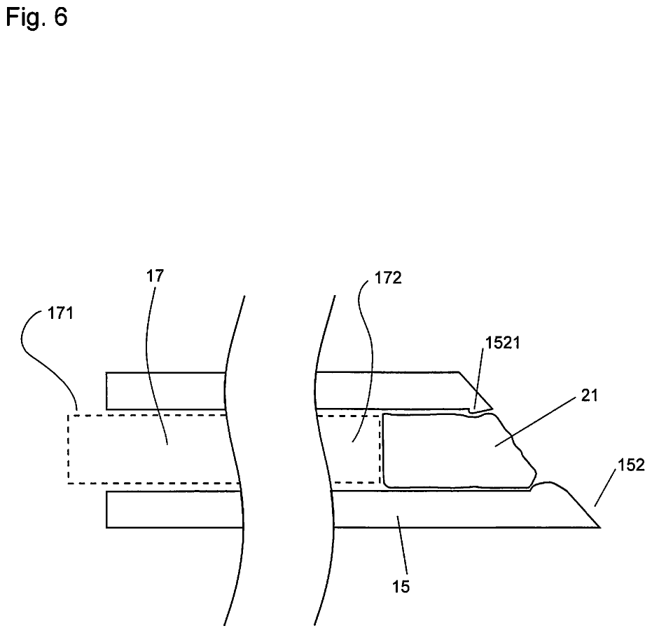

As presented in FIG. 6, hollow needle 15 is configured with a sharp tip 152 and one or more internal protrusions 1521, configured to retain a solid bone core 21 upon retraction of the needle tip from the bone 2. Elongated slider 17 may be configured to move proximally as new core material enters the needle. By way of example and not limitation, proximal side of bone core 21 may push distal end of elongated slider 17 freely when the needle tip enters the bone, until a point at which user desires to remove core from needle by pushing the proximal side 171 of the elongated slider distally with respect to the needle 15.

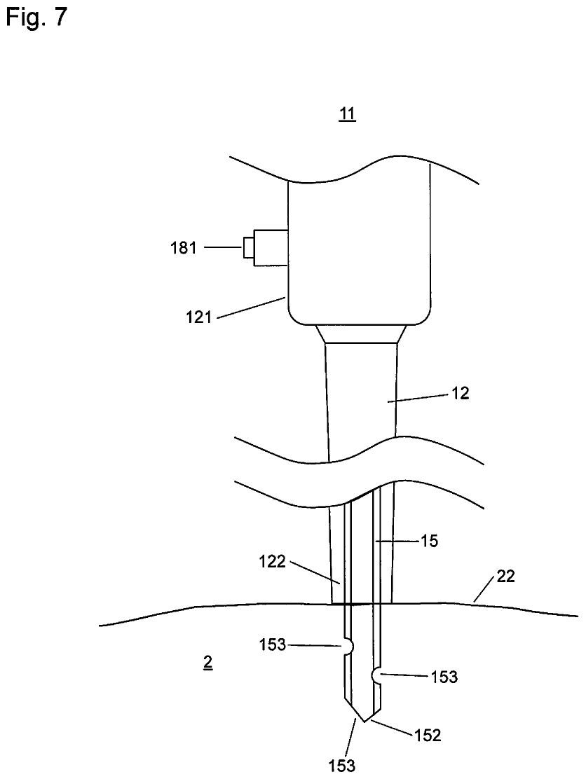

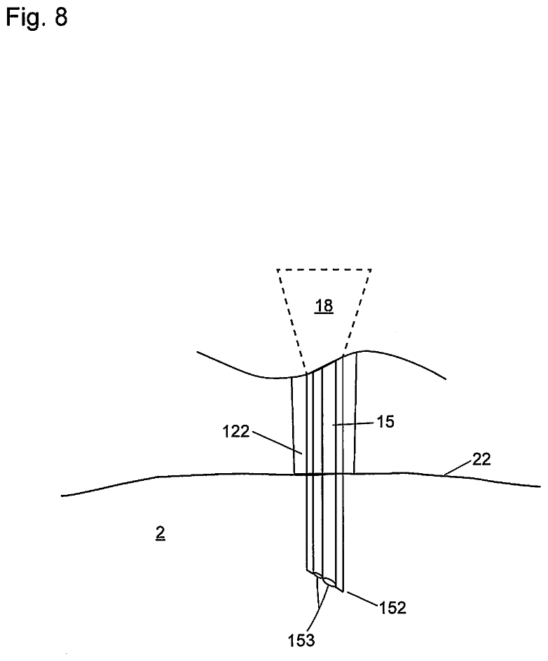

As presented in FIG. 7, a hollow penetrating needle 15 may be driven into bone 2 containing bone marrow, blood, stem cells, or other desirable fluids. Negative pressure may be achieved by attaching a tube to luer fitting or other adapter 181 on the device handle 11 or on the hollow shaft 12, and actuating a pump, syringe or other negative pressure element. In the embodiment shown, the adapter port is on the proximal portion 121 of the hollow shaft. Access points 153 for fluid to exit the bone and enter the hollow shaft may be located on the sides of the needle as shown in FIG. 7, or embodied by a multi-lumen tube as depicted in FIG. 8.

As presented in FIG. 8, A hollow needle 15 comprising a multi-lumen 153 tube is inserted into bone 2. A negative pressure is then applied through an instrument 18 down a cannula 122 past the bone surface 22 and ultimately to the bone cavity. In one embodiment, negative pressure 18 is applied to a first lumen to extract liquid blood and bone marrow, and a second lumen is utilized for core extraction. By way of example and not limitation, a pilot hole may be created using a removable metal penetrating attachment, and then a hard polymer multi-lumen tube may be inserted and advanced via biasing element for liquid blood, bone marrow, and bone extraction.

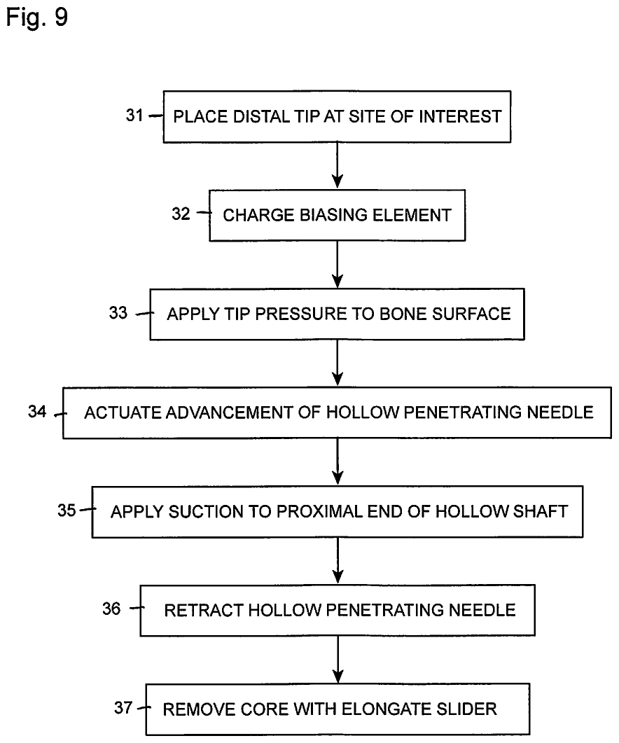

As presented in FIG. 9, the automated tissue coring device of the present invention may be utilized for bone marrow biopsies. In one embodiment, a bone marrow biopsy is performed with the following steps: 31 Place the distal tip at the site of interest; second 32 charge the internal spring (biasing element) by squeezing the lever (first actuator). 33 Apply tip pressure to the bone surface and actuate 34 first impact or advancement by squeezing the trigger (second actuator). Trigger impact or advancement again if desired depth is not yet achieved. Depth may be indicated by markings on the needle or via a window in the handle which shows how far the needle has moved from its starting position. Repeat triggering impact or advancement until desired depth is achieved. With the hollow shaft tip in place, pull back on the handle toggle to retract the needle 36 from the bone. Remove the penetrating needle with the core in it from the back of the device. Attach provided tight sealing fitting onto the adapter 181, sealing off the proximal-most section of the hollow shaft, and creating a continuous channel between the fitting and the end of the shaft. With the hollow shaft tip tightly in place at the entrance to the hole, apply suction 18 to the fitting in order to withdraw blood and bone marrow. A second hollow elongated body may be inserted through the back of the device before the fitting is attached in order to seal off the proximal section and taper down the distal end for a tight seal on the hole in the bone. Finally, extract the bone tissue core from the needle by pushing it through a wire opening with an elongated slider 37.

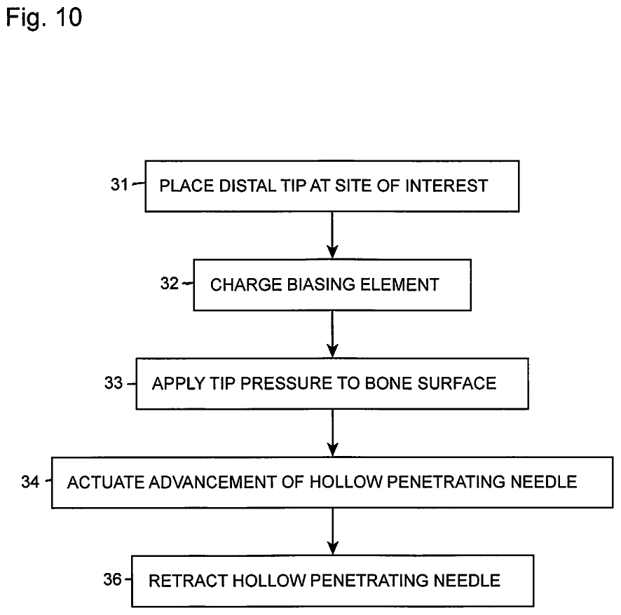

As presented in FIG. 10, the automated tissue coring device of the present invention may be utilized for bone marrow enhanced tissue repair. In one embodiment, bone marrow stimulation is achieved by performing the following detailed steps: 31 Place the distal tip at the site of interest; second 32 charge the internal spring (biasing element) by squeezing the lever (first actuator). 33 Apply tip pressure to the bone surface and actuate 34 first impact or advancement by squeezing the trigger (second actuator). Trigger impact or advancement again if desired depth is not yet achieved. Depth may be indicated by markings on the needle or via a window in the handle which shows how far the needle has moved from its starting position. Repeat triggering impact or advancement until desired depth is achieved. Once desired depth is achieved, engage the toggle on the handle to activate a retraction mode, and then squeeze the second actuator to retract the needle. When the actuator reaches the end of its stroke the toggle is pushed back into forward mode. Repeat from step 31 until desired number of holes are created in bone. Each new hole will drive cores from previous holes further proximally into the hollow penetrating needle. In an alternative embodiment an elongated slider is used after each coring operation to press out and dispose of the core in order to provide an unobstructed needle opening for the next coring operation.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

1. A tissue coring device comprised of:
a handle;
a hollow shaft within a device body;
a biasing element, said biasing element housed in the handle, said biasing element coupled to a linear advancement mechanism
a first actuator;
and a hollow penetrating needle; said first actuator operably connected to the hollow penetrating needle.

2. The tissue coring device of claim 1 wherein the penetrating needle has an angled tip.

3. The tissue coring device of claim 1 wherein a distal portion of the hollow shaft is curved for accessing a coring location.

4. The tissue coring device of claim 1 wherein the biasing element is operably connected to an indexing and an impactor mechanism.

5. The tissue coring device of claim 4 wherein the linear advancement mechanism of the biasing element may be selected from a linear indexing Geneva mechanism, a rack and pinion, a slider crank, a barrel cam and follower, a slotted bar quick return mechanism, a Whitworth mechanism, a vibrating penetrator, or an oscillating swash plate mechanism.

6. The tissue coring device of claim 1 further including a second actuator, said second actuator operably connected to the hollow penetrating needle for retraction.

7. The tissue coring device of claim 1 further including a toggle and a secondary mode for the first actuator.

8. The tissue coring device of claim 1 wherein the hollow shaft is removable from the handle and exchangeable with other attachments.

9. The tissue coring device of claim 1 wherein the hollow shaft includes a hollow tube with a port for suction and further including a first slider, said first slider including a cutting tip.

10. The tissue coring device of claim 1 further including an elongate slider and positioned within the hollow needle.

11. A method for bone marrow biopsies by using a tissue coring device, the method comprising;
placing a distal tip of a penetrating needle of the tissue coring device at the site of interest;
charging a biasing element by squeezing a first actuator;
applying tip pressure to a bone surface and actuating a first impact or advancement by squeezing a trigger of a second actuator;
monitoring a tip depth by markings on the needle or by a window in a handle of the tissue coring device;
repeating triggering impact or advancement until a desired depth is achieved;
placing a hollow shaft tip in place;
pulling back on the handle to retract the penetrating needle from the bone; and
removing the penetrating needle with a core from the device.

12. The method for bone marrow biopsies of claim 11 further comprising;
attaching a tight sealing fitting onto an adapter on the tissue coring device;
sealing off a proximal-most section of the hollow shaft, and creating a continuous channel between the fitting and the end of the shaft; and
applying suction to the fitting in order to withdraw blood and bone marrow.

13. The method for bone marrow biopsies of claim 12 further comprising;
inserting a second hollow elongated body through aback of the device before the fitting is attached; and
extracting a bone tissue core from the needle by pushing it through a wire opening with an elongated slider.

14. A method for a bone marrow enhanced tissue repair, the method comprising;
placing a distal tip of a penetrating needle of a tissue coring device at the site of interest;
charging a biasing element by squeezing a first actuator,
applying a tip pressure to a bone surface;
advancing the needle by squeezing a trigger of a second actuator;
repeating a triggering impact or advancement until a desired depth is achieved. engaging a toggle on a handle of the device to activate a retraction mode;
squeezing the second actuator to retract the needle; and
toggling a forward mode and repeat until desired number of holes are created in bone.
